(19) United States
(12) Patent Application Publication
(10) Pub. No.: US 2011/0066185 A1
(43) Pub. Date: Mar. 17, 2011

(54) Bone joint suture button and grommet and kit using same

(76) Inventor: Harold M. Wotton, III, Woodstock, CT (US)

(21) Appl. No.: 12/653,607

(22) Filed: Dec. 16, 2009

Related U.S. Application Data
(60) Provisional application No. 61/201,831, filed on Dec. 16, 2008.

(51) Int. Cl. A61B 17/04

(52) U.S. Cl. 606/228; 606/233

(57) ABSTRACT

An improved bone joint suture button including a body having a channel extending across at least a portion of the top surface of the body, a pair of suture passages extending from the channel inclined, inwardly to the bottom of the body, and a bridge portion recessed from the body top surface between the suture passages and having a curved upper surface for providing a smooth surface for bearing against the suture; and an improved bone joint suture grommet including a pair of grommets to be inserted into holes drilled in a bone, each grommet including a hollow shaft and a head portion having a top with a chamfered channel in communication with the interior of the hollow shaft, a pair of buttons each including a chamfered side wall, a top with a chamfered channel therein, and a pair of inwardly angled suture passages extending from the channel through the button and separated by a chamfered portion, and at least one suture strand looped through the pair of inwardly angled suture passages of one button over the chamfered portion and secured to a needle.

### RELATED APPLICATIONS

This application claims benefit of and priority to U.S. Provisional Application Ser. No. 61/201,831 filed Dec. 16, 2009, under 35 U.S.C. §§119, 120, 363, 365, and 37 C.F.R. §1.55 and §1.78 which is incorporated herein by this reference.

### FIELD OF THE INVENTION

This invention relates to an improved bone joint suture grommet, bone joint suture button and a bone joint suture kit using same.

### BACKGROUND OF THE INVENTION

Lateral suture techniques are used to repair a wide variety of bone joint problems in humans and animals such as dogs. In one of many applications a knee joint is repaired by drilling a hole from the lateral side of the femur to the medial side and another hole is drilled from the lateral side of the tibia to the medial side. A suture is inserted through both holes and then secured to stabilize the knee joint. Over time through cycles of joint movement two serious problems arise. The suture strands saw through the bone and/or the bone wears away and weakens the suture strands. Further, bone anchors used to secure the sutures may be large and irritate the soft tissue.

### SUMMARY OF THE INVENTION

It is therefore an object of this invention to provide an improved bone joint button, bone joint grommet, and a bone joint suture kit.

It is a further object of this invention to provide such an improved bone joint button and bone joint grommet which reduce wear on the sutures and the bone.

It is a further object of this invention to provide such an improved bone joint which are simple and easy to install and do not present sharp, prominent edges to soft tissue.

It is a further object of this invention to provide such an improved bone joint suturing kit which contains the basic parts to effect a bone joint suture operation.

It is a further object of this invention to provide such an improved bone joint button which is chamfered or tapered where it engages the hole in the bone for a self-centering and stabilizing the button in the hole.

The invention results from the realization of a suture grommet having a suture passage therethrough; a head having an opening therethrough to the suture passage and a drive surface configured to receive a drive tool to set the grommet in a hole in a bone joint; the opening having its inner edge chamfered to provide a smooth continuous surface for bearing against the suture and a suture button including a body having a channel extending across at least a portion of the top surface of the body; a pair of suture passages extending from the channel inclined, inwardly to the bottom of the body; a bridge portion recessed from the body top surface between the suture passages and having a curved upper surface for providing a smooth surface for bearing against the suture.

The subject invention, however, in other embodiments, need not achieve all these objectives and the claims hereof should not be limited to structures or methods capable of achieving these objectives.

This invention features a bone joint suture button including a body having a channel extending across at least a portion of the top surface of the body, a pair of suture passages extending from the channel inclined, inwardly to the bottom of the body, and a bridge portion recessed from the body top surface between the suture passages and having a curved upper surface for providing a smooth surface for bearing against the suture.

In a preferred embodiment each suture passage may be chamfered along at least a part of its inner edge to provide smooth surfaces for bearing on the suture. Each chamfered edge may be radiused. Each body may be round. The lower edge of the body may be chamfered for a self-centering and stabilizing the body in a hole in the bone. The edge of the top surface of the body may be chamfered to avoid irritation of soft tissue. This invention also features a bone joint suture grommet including a shaft having a suture passage therethrough, a head having an opening therethrough to the suture passage and drive surface configured to receive a drive tool to set the grommet in a hole in a bone joint, the opening having its inner edge chamfered to provide a smooth continuous surface for bearing against the suture.

In preferred embodiment the chamfer may extend completely around the inner edge. The chamfered inner edge may be radiused. The drive surface may be on the outer edge of the head and may form a drive nut. The drive surface may include a drive member extending within and across the opening. The shaft may include external threads.

This invention also features a bone joint suture kit including a pair of grommets to be inserted into holes drilled in a bone, each grommet including a hollow shaft and a head portion having a top with a chamfered channel in communication with the interior of the hollow shaft, a pair of buttons each including a chamfered side wall, a top with a chamfered channel therein, and a pair of inwardly angled suture passages extending from the channel through the button and separated by a chamfered portion, and at least one suture strand looped through the pair of inwardly angled suture passages of one button over the chamfered portion and secured to a needle.

In preferred embodiments the kit may further include a second suture strand looped through the pair of inwardly suture passages of the second button over the chamfered portion thereof and secured to a second needle. The head portion may be configured to receive a driver for setting the grommet into a hole in a bone. The hollow shaft may include external threads. The kit may further include a tap for forming internal threads into holes drilled in the bone and may further include a driver for engaging the head portion.

### BRIEF DESCRIPTION OF THE SEVERAL VIEWS OF THE DRAWINGS

Other objects, features and advantages will occur to those skilled in the art from the following description of a preferred embodiment and the accompanying drawings, in which:

FIG. 1 is a diagrammatic, three dimensional view of a knee joint using one embodiment of the suture buttons and grommets according to this invention showing the grommets installed on the lateral side of the tibia and femur;

FIG. 2 is side diagrammatic, three dimensional view of the knee of FIG. 1 from the medial side showing the suture button installed on the medial side of the tibia and femur according to this invention;

FIG. 3 is a three dimensional view of one embodiment of a suture grommet according to this invention;

FIG. 4 is a side sectional, elevational view of the grommet of FIG. 3;

FIG. 5 is a top plan view of the head of the grommet of FIG. 3 configured to receive an external drive tool;

FIG. 6 is a three dimensional view of a drive tool for engaging the grommet head of FIG. 5;

FIG. 7 is a top plan view of another embodiment for the grommet head with a different, internal drive configuration;

FIGS. 8 and 9 are two different drive tools that may be used to engage with the grommet head of FIG. 7;

FIG. 10 is a three dimensional view of one embodiment of the suture button according to this invention;

FIG. 11 is a side plan view of the button of FIG. 10;

FIG. 12 is a top sectional view along lines 12-12 of FIG. 11;

FIG. 13 is a side sectional view along lines 13-13 of FIG. 11;

FIG. 14 is a side sectional view of a bone anchor that could be used with this invention;

FIG. 15 is a side elevational view of a tap that can be used in the kit of this invention;

FIG. 16 is a three dimensional view of a button engaged with a loop of suture strands and led by a needle used in the kit of this invention;

FIG. 17 is a side sectional diagrammatic view of a bone suture operation using this invention;

FIG. 18 is a view similar to FIG. 17 of another bone joint suture operation according to this invention;

FIG. 19 is a view similar to FIG. 17 of still another bone joint suture operation according to this invention; and

FIG. 20 is a view similar to FIG. 17 of yet another bone joint suture operation according to this invention.

### DETAILED DESCRIPTION OF THE INVENTION

Aside from the preferred embodiment or embodiments disclosed below, this invention is capable of other embodiments and of being practiced or being carried out in various ways. Thus, it is to be understood that the invention is not limited in its application to the details of construction and the arrangements of components set forth in the following description or illustrated in the drawings. If only one embodiment is described herein, the claims hereof are not to be limited to that embodiment. Moreover, the claims hereof are not to be read restrictively unless there is clear and convincing evidence manifesting a certain exclusion, restriction, or disclaimer.

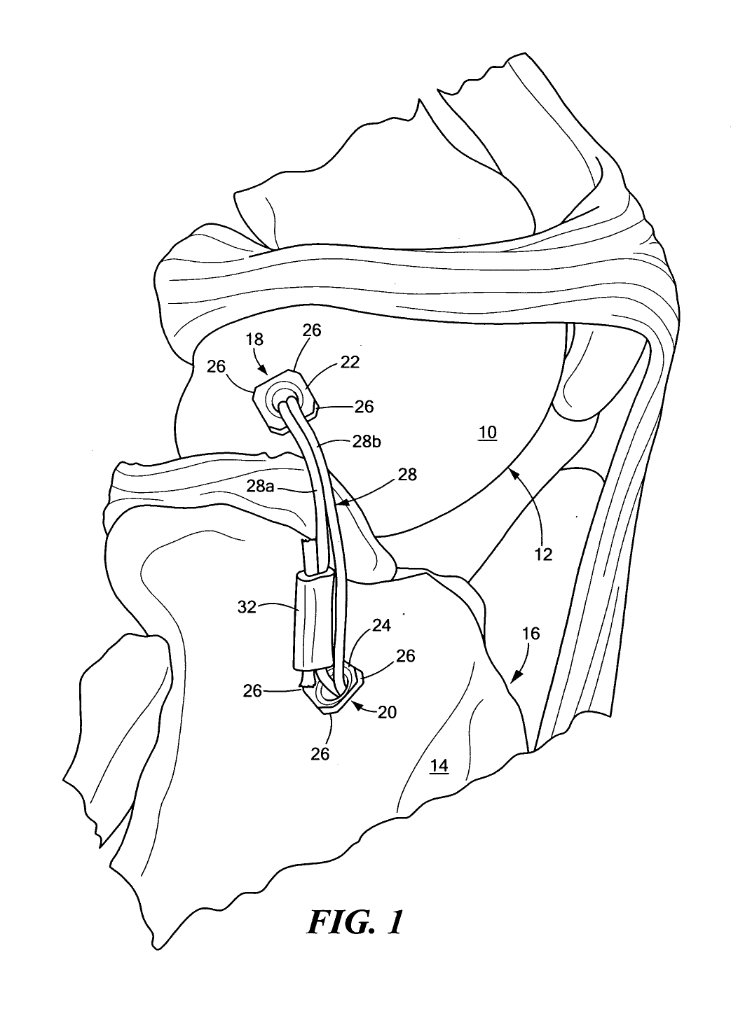

There is shown in FIG. 1 the lateral side 10 of a femur 12 and the lateral side 14 of a tibia 16 which have been joined together using one embodiment of the suture buttons, grommets, and kit according to this invention. A hole is drilled through femur 12 from the lateral side 10 and tibia 16 through the lateral side 14. Suture grommets 18 and 20 having heads 22 and 24, respectively, are inserted in those holes, for example, by force fitting or they may be threaded and the holes may be tapped to threadably receive them. Heads 22 and 24 may have their corners rounded off as at 26 to prevent irritation of soft tissue. A single suture 28 having suture strands 28a and 28b extend from the proximal ends at the heads 18 and 20 and are joined by crimp 32.

On the medial side 36 of tibia 16, and medial side 38 of femur 12, FIG. 2, suture buttons 40 and 42 receive loops of suture strands 28a and 28b. Suture 28 is one continuous piece looped around buttons 40 and 42 and appears as two strands 28a and 28b in FIG. 1. As a part of the method of installation the strands 28a and 28b of suture 28 are pulled tight so that suture buttons 40 and 42 are pulled tight to the bone, then the tension is set on the strands and they are crimped using crimp 32.

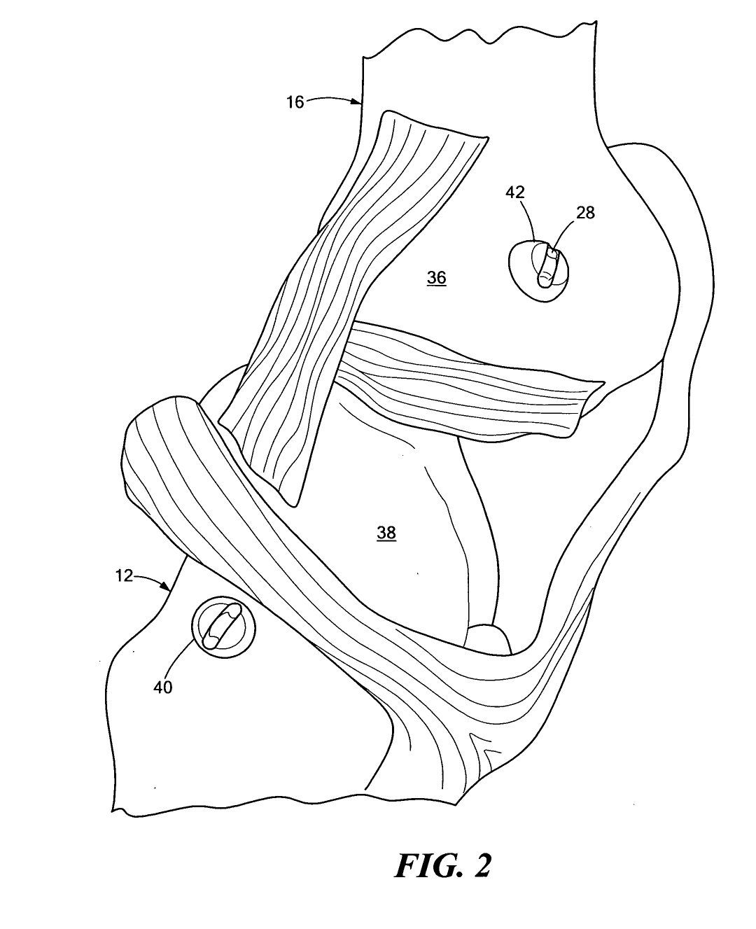

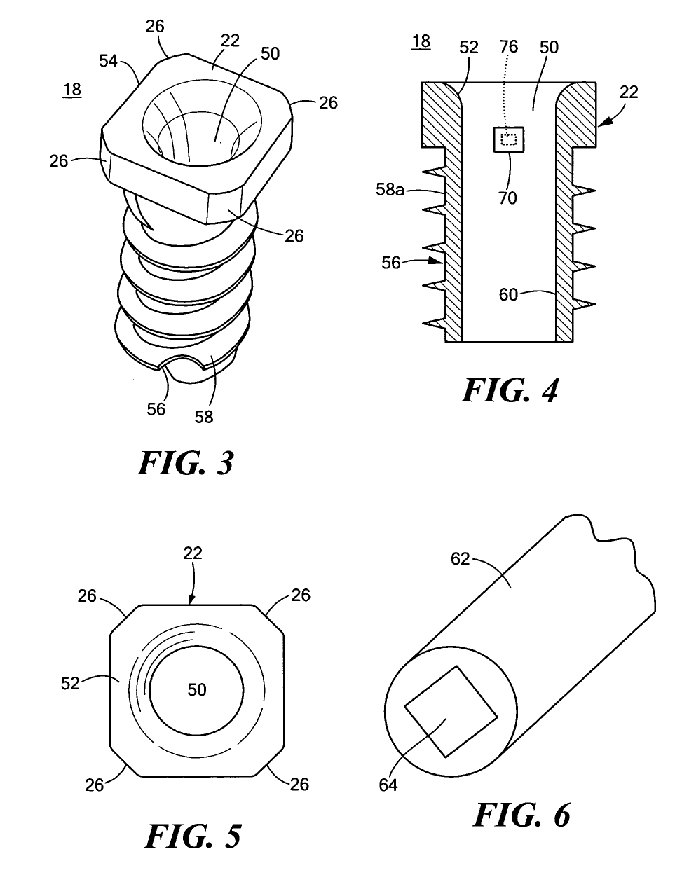

Each grommet 22, 24 as exemplified by grommet 22, FIG. 3, includes a head 22 containing a hole 50 which is chamfered as at 52 on the upper edge to remove the stress on the suture. Corners 26 are rounded to reduce an irritation with soft tissue and the upper edge 54 may also be chamfered or radiused for the same purpose. Hole 50 communicates with a suture passage through shaft 56 which may be smooth for a force fitting or may have threads as shown at 58 for engaging threads that are tapped in the hole in the bone.

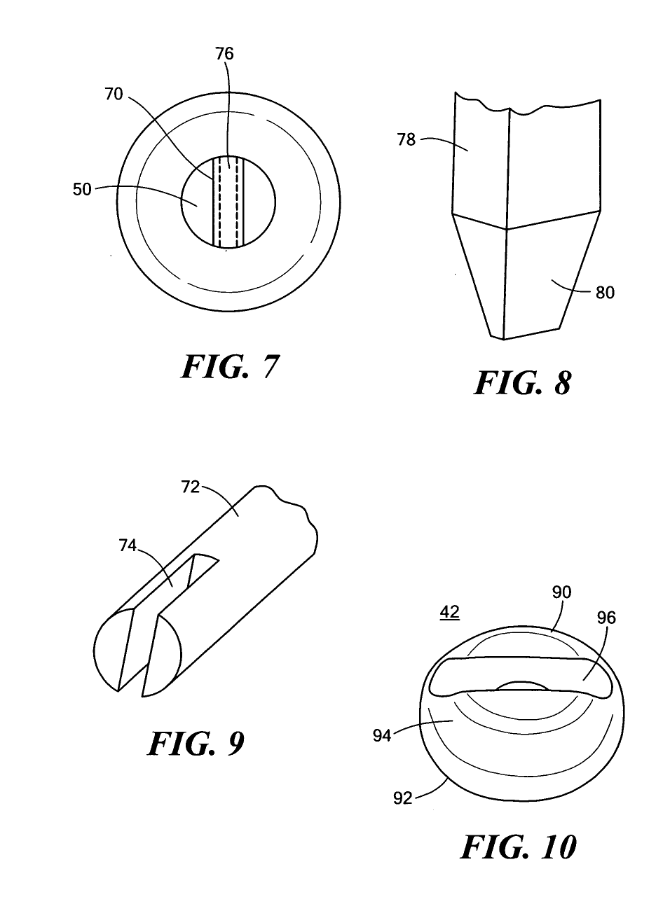

Suture grommet 18 is shown in diagrammatic side sectional view in FIG. 4 where the threads on shaft 56 have been eliminated and made smooth sided 58a for force fitting. Shaft 56 has a suture passage 60 through it which communicates with the hole 50 in head 22. The chamfered inner edge 52 of hole is here shown more clearly as is its rounded or radiused profile. Head 22 may have its outer surface or periphery configured in a square or square-like shape in the form of a nut, as apparent in FIG. 5, for engaging with a drive tool 62, FIG. 6. Drive tool 62 contains square socket 64 for engaging head 22 and rotating or force fitting grommet 18 into the hole drilled in the bone. Head 22 can be configured with any desired shape, e.g. square, hexagonal, octagonal, to engage with the driving tool. The configuration of head 22 to engage a driving tool is not limited to an external shape: it may be internal as well. For example, there may be a drive member 70, FIG. 7, extending within and across hole 50 to be gripped by slotted tool 72, FIG. 9, having a slot in one end 74 for gripping drive member 70. Alternatively, drive member 70 may have a slot 76 running along it which is engageable by a tool 78, FIG. 8, having a narrow bit 80 in the nature of a screwdriver. Drive member 70 is shown in phantom in FIG. 4.

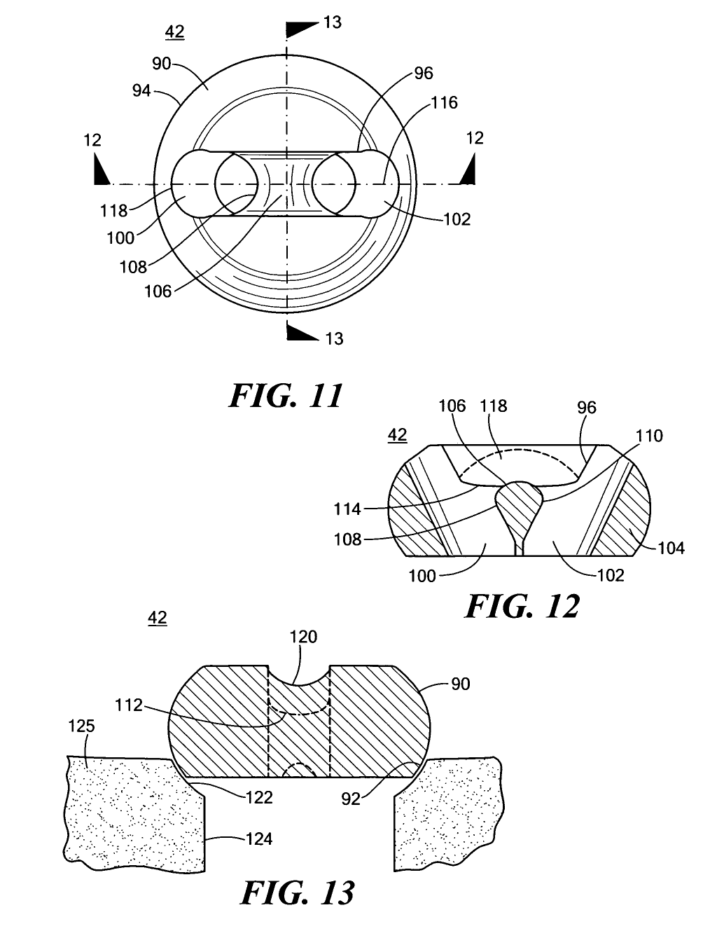

Suture buttons 42 and 44 as exemplified by suture button 42, FIG. 10, has a chamfered top edge 90 and bottom edge 92 resulting in a generally convex side wall 94. The chamfering or radiusing reduces the irritation of soft tissue. The lower chamfer or radiusing 92 is used to self center and stabilize the position of button 42 in the chamfered end of the hole drilled in the bone. Button 42 also includes a channel 96 which can be seen more clearly in FIGS. 11, 12 and 13. In FIG. 11 the top view shows channel 96 extending across the top of button 42 and interconnecting with two suture passages 100 and 102. Suture passages 100 and 102, FIG. 12, are inclined inwardly to converge within the body 104 of suture button 42. Center portion 106, FIGS. 11 and 12 may be radiused at both sides 108, 110, again to protect the suture from wear and fraying. Channel 96 may be concave across its narrow dimension 112, FIG. 13. Channel 96 may be flat along its longitudinal extent as shown at 114 or it may be convex as shown at 116 and increasing in radius as at 118 in the center portion 106 and then reducing again to the smaller radius at 118 at the other end. If that convex contour is used then the profile as shown in FIG. 13 will reflect the upper convex area along the channel with the concavity at 120. The chamfer at 92 here shown as a radius nests snuggly in the chamfer 122 at the top of hole 124 drilled in the bone 125. The mating of the chamfer 92 straight or radiused as shown and the chamfer 122 in hole 124 serves not only to automatically center suture button 42 in hole 124 but it also stabilizes it and keeps it from wobbling back and forth.

Although bone screws or anchors are no longer necessary when using this invention a bone anchor can be used if still desired. Such a bone anchor 130 is shown in

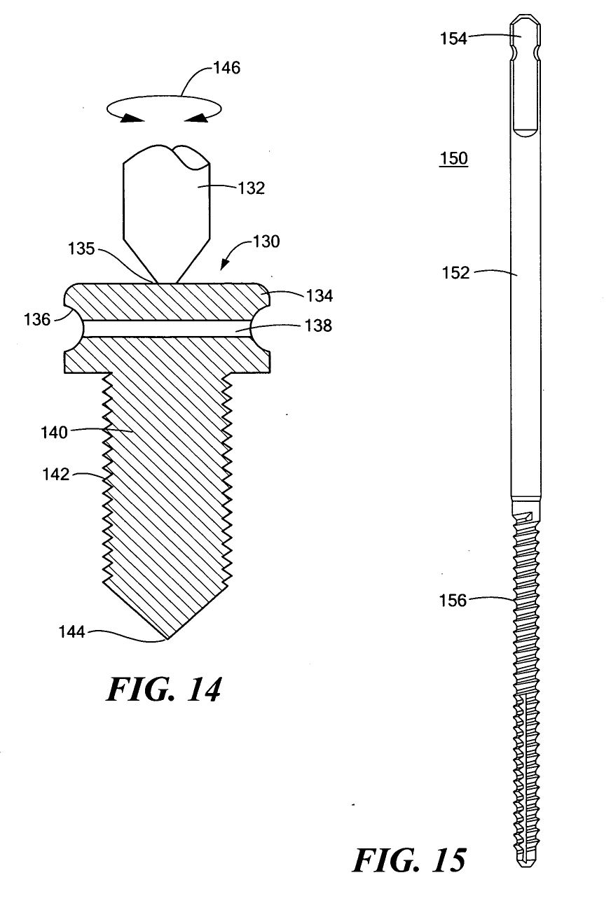

FIG. 14 with an integral drive bit 132 attached to it. Bone anchor 130 includes a head 134 with a spindle portion 136 and a through hole 138. There is also a shaft 140 with threads 142. In use, bit 132 is gripped in the chuck of a drill, for example, and the point 144 of bone anchor 130 is driven into the bone using bit 132 to rotate bone anchor 130. When it is fully set, bit 132 can be wiggled back and forth as indicated by arrow 146 to cause it to snap off from head 134 at 135. Before bone anchor 130 is driven into the bone the sutures are led in and through through hole 138 so that as the bone anchor is driven in to the bone the sutures are wound on spindle 136 to the desired tightness.

A tapping tool 150, FIG. 15, can be included in the kit according to this invention. Tapping tool 150 is a conventional tool with a shank 152 whose upper portion 154 is gripped in a drive tool and whose lower portion 156 contains the tapping threads. Also included in the kit according to this invention is a needle 160, the end of which is connected at 163 to suture strands 164 and 166 of suture 162. The other end of suture 162 forms a loop 168 which is engaged with the holes 100 and 102, FIGS. 11-13, of button 42a.

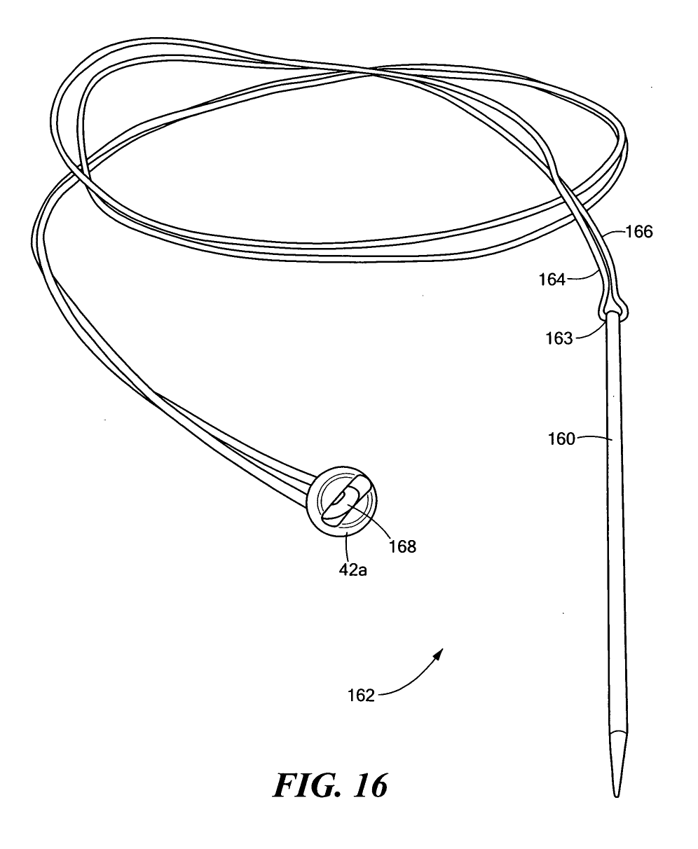

The kit according to this invention may include one or more needles 160, and sutures 162 and button 42a as shown in FIG. 16, as well as an additional button 42, two suture grommets 18 and 20, a tap 150, FIG. 15, and a drive tool such as drive tool 62, FIG. 6; drive tool 78, FIG. 8, or drive tool 72, FIG. 9.

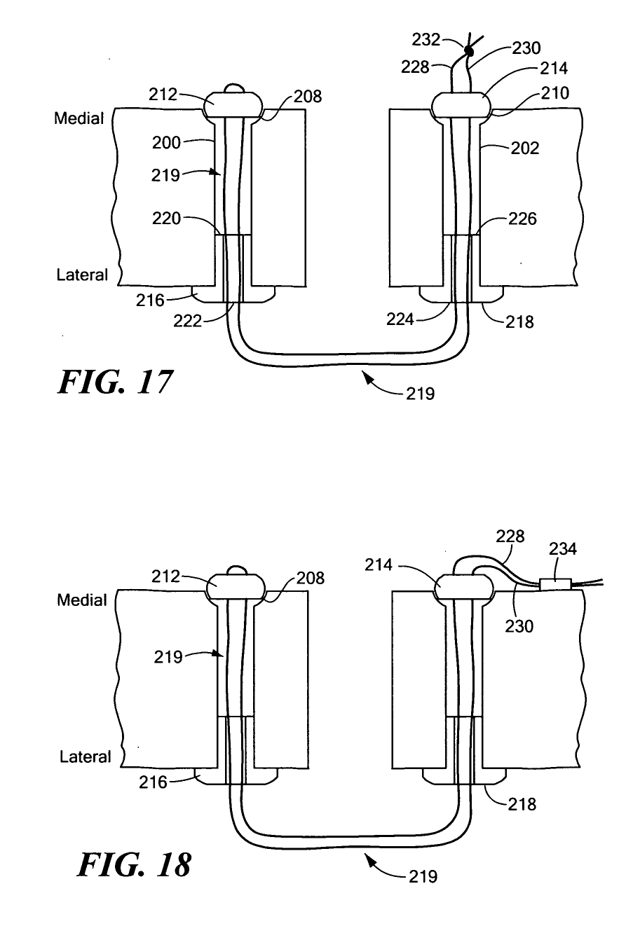

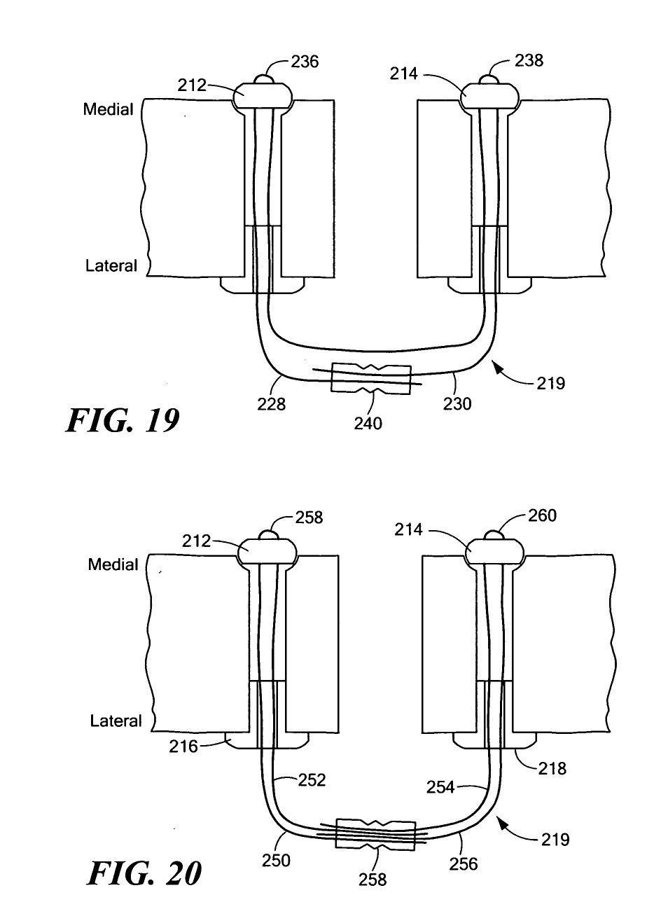

The apparatus of this invention may be used, FIG. 17, by drilling the hole 200, 202 in each bone 204, 206 shown only schematically in FIG. 17. The holes may be chamfered at their upper ends 208, 210 to receive the chamfered portion of suture buttons 212, 214. At the medial end of each hole a suture grommet 216, 218 is set into the lateral side of each hole, 200, 202, either by force fitting or threading using a tap in holes 200, 202 or a similar device. Then suture 219 is looped about suture button 212, fed through the distal end 220 of suture grommet 216 and then out the proximal end 222 of suture grommet 216. This is then led into the proximal end 224 and out the distal end 226 of grommet 218, which is also set in the lateral side of bone 206. Both suture strands 228, 230 are fed through button 214 and then tied with a knot for example 232 to draw the suture strands up tight and nest suture buttons 212 and 214 tightly against bones 204 and 206, respectively. In another approach, FIG. 18, suture strands 228 and 230 are led through a bone anchor 234 after been looped around suture button 212, fed through suture grommets 216 and 218 and through suture button 214. In still another approach, suture 219, FIG. 19, is looped 236 through button 212 and also looped 238 through button 214. The loose ends of strands 228 and 230 are then crimped using a crimp device 240 and crimping tool to secure strands 228 and 230. In yet another approach, FIG. 20, the suture 219 is composed of two sets of strands 250, 252 and 254, and 256 each of which is looped 258, 260 about buttons 212, 214, respectively, then run through suture grommets 216 and 218 where upon all four strands 250-256 are secured by crimp 258. In FIGS. 17-20 the bones and joints are shown only schematically for clarity and the sutures and suture strands are shown loose but of course in the actual use they would be drawn up snugly to the tension required to repair the joint.

Although specific features of the invention are shown in some drawings and not in others, this is for convenience only as each feature may be combined with any or all of the other features in accordance with the invention. The words “including”, “comprising”, “having”, and “with” as used herein are to be interpreted broadly and comprehensively and are not limited to any physical interconnection. Moreover, any embodiments disclosed in the subject application are not to be taken as the only possible embodiments.

In addition, any amendment presented during the prosecution of the patent application for this patent is not a disclaimer of any claim element presented in the application as filed: those skilled in the art cannot reasonably be expected to draft a claim that would literally encompass all possible equivalents, many equivalents will be unforeseeable at the time of the amendment and are beyond a fair interpretation of what is to be surrendered (if anything), the rationale underlying the amendment may bear no more than a tangential relation to many equivalents, and/or there are many other reasons the applicant can not be expected to describe certain insubstantial substitutes for any claim element amended.

Other embodiments will occur to those skilled in the art and are within the following claims.

What is claimed is:

1. A bone joint suture button comprising:
a body having a channel extending across at least a portion of the top surface of said body;
a pair of suture passages extending from said channel inclined, inwardly to the bottom of said body; and
a bridge portion recessed from said body top surface between said suture passages and having a curved upper surface for providing a smooth surface for bearing against the suture.

2. The bone joint suture button of claim 1 in which each said suture passage is chamfered along at least a part of its inner edge to provide smooth surfaces for bearing on the suture.

3. The bone joint suture button of claim 2 in which each said chamfered edge is radiused.

4. The bone joint suture button of claim 1 in which each said body is round.

5. The bone joint suture button of claim 1 in which the lower edge of said body is chamfered for a self-centering and stabilizing the body in a hole in the bone.

6. The bone joint suture button of claim 1 in which the edge of said top surface of said body is chamfered to avoid irritation of soft tissue.

7. A bone joint suture grommet comprising:
a shaft having a suture passage therethrough;
a head having an opening therethrough to said suture passage and drive surface configured to receive a drive tool to set said grommet in a hole in a bone joint;
said opening having its inner edge chamfered to provide a smooth continuous surface for bearing against the suture.

8. The bone joint suture grommet of claim 7 in which said chamfer extends completely around said inner edge.

9. The bone joint suture grommet of claim 7 in which said chamfered inner edge is radiused.

10. The bone joint suture grommet of claim 7 in which said drive surface is on the outer edge of said head and forms a drive nut.

11. The bone joint suture grommet of claim 7 in which said drive surface includes a drive member extending within and across said opening.

12. The bone joint suture grommet of claim 7 in which said shaft includes external threads.

13. A bone joint suture kit comprising:
a pair of grommets to be inserted into holes drilled in a bone, each grommet including a hollow shaft and a head portion having a top with a chamfered channel in communication with the interior of the hollow shaft;
a pair of buttons each including a chamfered side wall, a top with a chamfered channel therein, and a pair of inwardly angled suture passages extending from the channel through the button and separated by a chamfered portion; and
at least one suture strand looped through the pair of inwardly angled suture passages of one button over the chamfered portion and secured to a needle.

14. The kit of claim 13 further including a second suture strand looped through the pair of inwardly angles suture passages of the second button over the chamfered portion thereof and secured to a second needle.

15. The kit of claim 13 in which said head portion is configured to receive a driver for setting said grommet into a hole in a bone.

16. The kit of claim 13 in which said hollow shaft includes external threads.

17. The kit of claim 16 further including a tap for forming internal threads into holes drilled in the bone.

18. The kit of claim 15 further including a driver for engaging said head portion.
